(19) United States
(12) Patent Application Publication
(10) Pub. No.: US 2005/0113443 A1
(43) Pub. Date: May 26, 2005

(54) Modulation of airway inflammation in patients with cystic fibrosis and related diseases

(76) Inventors: Christopher L. Karp, Cincinnati, OH (US); Nicos A. Petasis, Hacienda Heights, CA (US)

Correspondence Address: FISH & RICHARDSON, PC, 12390 EL CAMINO REAL, SAN DIEGO, CA, 92130-2081, US

(21) Appl. No.: 10/858,840

(22) Filed: Jun. 1, 2004

Related U.S. Application Data
(60) Provisional application No. 60/539,820, filed on Jan. 27, 2004.
(60) Provisional application No. 60/475,963, filed on Jun. 1, 2003.

(51) Int. Cl. A61K 31/38; A61K 31/202; A61K 31/335

(52) U.S. Cl. 514/450; 514/560

(57) ABSTRACT

This invention provides a method for treating pulmonary disease in patients with cystic fibrosis, variant cystic fibrosis, and non-cystic fibrosis bronchiectasis. The method involves administering a pharmaceutically effective amount of a lipoxin or lipoxin analogue to subjects with cystic fibrosis or related disease, in amounts sufficient to downregulate harmful neutrophilic airway inflammatory responses.

### RELATED APPLICATIONS

Priority is claimed herein to U.S. provisional patent application Nos. 60/475,963, to Karp et al., filed Jun. 1, 2003, entitled “Methods And Compositions For The Modulation Of Airway Inflammation In Patients With Cystic Fibrosis” and Ser. No. 60/539,820, Karp et al., filed Jan. 27, 2004, entitled “Modulation Of Airway Inflammation In Patients With Cystic Fibrosis and Related Diseases.” The disclosure of the above-referenced applications is incorporated by reference herein in its entirety.

### FIELD OF THE INVENTION

The present invention relates generally to pharmaceutical compositions and methods of use thereof for the prevention, amelioration and treatment of pulmonary disease in cystic fibrosis and related diseases, including variant cystic fibrosis, and non-cystic fibrosis bronchiectasis.

### BACKGROUND OF THE INVENTION

Cystic fibrosis (CF) is a common, lethal autosomal recessive disorder caused by mutations in the gene encoding the CF transmembrane conductance regulator (CFTR) [Fishman' Pulmonary Diseases and Disorders, Vol. 1, 803-824, McGraw-Hill, New York, 1998; Cystic Fibrosis, 2649 2680, McGraw-Hill, New York, 1989]. The major clinical manifestations of CF include exocrine pancreatic insufficiency, male infertility, and chronic pulmonary disease. Chronic pulmonary disease, remains the major cause of morbidity and mortality in CF. Despite the molecular insights afforded by identification of CFTR, a clear understanding of the pathogenesis of lung disease in CF has remained elusive.

The CF airway is marked by chronic bacterial colonization and persistent neutrophilic inflammation. Bacterial colonization of the airways (with S. aureus, H. influenzae, and/or E. coli) generally occurs within the first year or two after birth. CF is associated with a special predisposition to subsequent colonization with Pseudomonas aeruginosa, an organism whose presence in the CF lung is associated with progressive respiratory compromise. By adulthood, 80-90% of patients with CF suffer from chronic pulmonary infection with mucoid strains of P. aeruginosa. Airway infection is associated with an exuberant inflammatory response dominated by neutrophils and the potent inflammatory mediators that are released by activated neutrophils. The end result of this mix of infection and dysregulated inflammation is progressive, bronchiectatic destruction of the lung.

Dysregulated, proinflammatory pulmonary immune responses go hand in hand with airway colonization and infection. There is a growing consensus that the CF airway is marked by an aberrant proinflammatory diathesis. In addition to the sustained presence of activated neutrophils and neutrophil-derived secretory products, there is significant upregulation of proinflammatory cytokine production: IL-8, TNF-α, and IL-1β are all markedly elevated in bronchoalveolar lavage fluid, sputa, and bronchial biopsies from patients with CF (Am J Respir Crit Care Med 152:2111, 1995; J Infect Dis 175:638, 1997; Eur Resp J 14:1136, 1999). This aberrant proinflammatory diathesis, demonstrable in a variety of in vitro and in vivo models, appears to predate infection in patients (J infect Dis 175:638, 1997; Am J Respir Crit Care Med 151:1075, 1995; Ped Pulm 20:63, 1995). In vivo studies employing fetal human tracheal grafts suggest strongly that the basal proinflammatory predisposition of the CF airway leads to the development of severe mucosal damage upon infection, damage that is integral to subsequent persistent bacterial colonization of the airway (Am J Respir Cell Mol Biol 23:121, 2000). An inflammatory diathesis thus appears to predate infection and secondarily impairs local host defenses in ways that are likely to promote bacterial colonization of the airway. Other evidence that the inflammatory response in CF isn't merely an appropriate adaptive response to ongoing bacterial infection is more indirect, if still compelling: steroids and nonsteroidal antiinflammatory drugs appear to preserve lung function in patients with CF without, however, increasing the pulmonary infectious burden (New Engl J Med 332:848, 1995; Cochrane Database Syst Rev 2:CD000407). Further, transgenic C5a receptor knockout mice, like patients with CF, fail to clear P. aeruginosa from the lung despite vigorous neutrophilic infiltration, suggesting that abnormal regulation of neutrophil trafficking and activation may be important to decreased bacterial clearance and excessive inflammatory responses (Nature 383, 86-9, 1996).

Unlike asthma and related chronic airway inflammation, which is characterized by the infiltration of T lymphocytes and eosinophils, the airway inflammatory response in CF is persistently neutrophilic, marked by: (a) upregulation of neutrophil chemotactic mediators such as IL-8; (b) florid accumulation of neutrophils in the airways; and (c) neutrophil activation, with abundant release of toxic products, such as neutrophil elastase. Paradigmatically, the initial inflammatory response to most bacterial stimuli, in the lung and elsewhere, is “acute”, i.e. neutrophil dominated. In the absence of clearance there is normally modulation over time to less histotoxic, “chronic” inflammation, a shift normally marked by the presence and immunoregulatory activity of monocytic cells and lymphocytes (Fundamental Immunology, Fourth Edition, 1051-1066, Lippincott-Raven, Philadelphia, 1999). An unusual feature of inflammation in the CF airway is that such modulation and shift to chronic inflammation never takes place. The CF airway remains dominated by neutrophilic inflammation. Two caveats should be mentioned: (a) this is not completely unique to CF; non-CF bronchiectasis is also marked by chronic or recurrent neutrophilic inflammation; and (b) aspects of chronic inflammation, especially lymphocyte infiltration, are apparent beneath the surface epithelium in CF (Clin Exp Immunol 124, 69, 2001).

The mechanisms responsible for dysregulated inflammation in the CF lung have remained unclear. The initial focus on the role of CFTR in regulating epithelial ion transport has provided a compelling account of the pathogenesis of gastrointestinal disease in CF as well as of the genesis of such CF-associated phenomena as high sweat NaCl content. However, the examination of altered ion and water transport alone has failed to clearly illuminate the path from gene to pathogenesis in the CF lung. However, In addition to direct chloride transport by CFTR, ion transport mediated by amiloride sensitive sodium channels, outwardly rectifying chloride channels, and potassium channels are altered in cells with mutant CFTR (Physiol Rev 79, S145,1999). CFTR is also thought to conduct ATP (Physiol Rev 79, S145,1999). More widely, a variety of cellular processes, including membrane recycling, protein processing, signal transduction pathways, and the secretion of immune mediators appear to be influenced by CFTR (Physiol Rev 79, S175, 1999; J Clin Invest 106, 403, 2000; J Immunol 164, 3377, 2000; Am J Respir Cell Mol Biol 23, 121, 2000). In turn, the expression and/or function of CFTR is modified by a variety of factors, including inflammatory mediators, hormones, signaling pathways, extracellular conditions, and pharmacological agents (Physiol Rev 79, S145,1999; Am J Physiol 267, C1398, 1994; Am J Physiol 272, L844, 1997; J Biol Chem 267, 16056, 1992; Eur Respir J 15, 937, 2000). The nexus of interaction with CFTR is thus thought to be wide, with multiple gene products upstream and downstream of CFTR. Circumstantial evidence for the likely role of at least part of the broad array of interacting gene products in the pathogenesis of inflammation and infection in the lung is provided by the poor correlation of CFTR genotype with phenotype in CF pulmonary disease (as opposed to pancreatic disease) (Clin Chest Med 19, 443,1998). Indeed, patients with variant CF (with chronic lung disease) who lack mutations in CFTR, and in whose families haplotype analysis reveals no linkage to CFTR, are well-recognized (N Engl J Med 347,401, 2002).

Despite a number of therapeutic approaches to CF in the clinic or under development, there remains a need for an effective method for modulating aberrant airway inflammatory responses in CF. While close attention to pulmonary toilet and vigorous treatment of infection with anti-bacterial agents have improved the clinical outlook for patients with CF, the norm is still an inexorable decline in pulmonary function, leading eventually to death or a need for lung transplantation. Non-specific inhibitors of inflammation have been tried as therapies for CF. Both alternate-day-prednisone therapy and therapy with high dose ibuprofen have been shown to retard the decrease in lung function in patients with CF. Potential side effects of such therapy have limited the usefulness of such general approaches, however. Other methods involving protein and gene augmentation therapy or the use of agents that suppress CF-associated stop mutations are also being developed, but are still at an experimental stage. Similarly, there remains a need for an effective method for suppress aberrant airway inflammatory responses in variant CF, and non-CF bronchiectasis.

Lipoxins (“lipoxygenase interaction products”[LX]) are trihydroxytetraene-containing arachidonic acid (AA; C20:4) metabolites that are important regulators of neutrophilic inflammation. Generated largely transcellulary (e.g. via interactions between airway epithelial cells and neutrophils), LX are functionally distinguishable from most other eicosanoid mediators (or modulators) of inflammation because of their potent antiinflammatory actions (Ernst Schering Res Found Workshop 31:143, 2000; Prostaglandins 53:107, 1997; Braz J Med Biol Res 34, 555,2001; Prostaglandins & other Lipid Mediators 68-69:433, 2002).

Three different biosynthetic pathways for lipoxin generation have been reported (Ernst Schering Res Found Workshop 31:143, 2000; Prostaglandins 53:107, 1997; Braz J Med Biol Res 34, 555,2001; Prostaglandins & other Lipid Mediators 68-69:433, 2002). All lead to the insertion of molecular oxygen at three sites in AA by a variety of different enzymes (5-, 15-, and 12-lipoxygenase (LO); and COX-2) that are generally segregated in different cell types and subject to regulation by cytokines and other inflammatory stimuli. These pathways likely operate both independently and in a co-regulated fashion in different tissues and biological situations.

In the first pathway, 15-LO in airway epithelial cells or monocytes (or neutrophils in some inflammatory conditions) generates 15S-hydroperoxyeicosatetraenoic acid (15S-H(p)ETE), or its reduced alcohol form 15S-hydroxyeicosatetraenoic acid (15S-HETE), through insertion of molecular oxygen (predominantly in the S configuration) into carbon 15 of AA. AA and its oxygenation products can transfer from cell to cell. Accordingly, 5S-H(p)ETE and 15S-HETE generated by the above cells serve as substrates for neutrophil 5-LO, leading to the generation of 5S,6S,15S-epoxytetraene, and subsequently the LXs, LXA4 and LXB4. These LXs maintain their carbon-15 alcohol in their precursor's S configuration. In the second pathway. 5-LO in myeloid cells (including neutrophils and monocytes) generates LTA4 from AA (via 5-H(p)ETE and 5-HETE). LTA4 has several potential fates: (a) nonenzymatic hydrolysis; (b) conversion to the proinflammatory leukotrienes LTB4 or LTC4; or (c) conversion, transcellularly to LXA4 or LXB5 by 15-LO in epithelial cells or 12-LO in platelets (through the same 5S,6S,15S-epoxytetraene of the first pathway). Finally, in the third, most recently-described, pathway (Proc Natl Acad Sci U S A 92, 9475, 1995), aspirin-induced acetylation of epithelial and endothelial COX-2 shuts off prostanoid synthesis and switches its catalytic activity to the generation of 15R-HETE (the stereoisomer of the above 15S-HETE) from AA. In turn, 15R-HETE released from such cells is transformed by neutrophil 5-LO into 15-epi-LXA4 and 15-epi-LXB4, stereoisomers of LXA4 and LXB4, respectively, with similar activities but greater functional potency due to their relative resistance to metabolic inactivation. Of note, cytochrome P450-dependent oxygenation of AA, an activity present in airway epithelial cells, leads to the generation of 15R-HETE in the absence of aspirin (Mol Med 2, 583, 1996).

The in vitro and in vivo activities of the best-characterized lipoxin, LXA4, include: (a) inhibition of neutrophil chemotaxis, adherence, and transmigration; (b) suppression of neutrophil activation (including NF-kB activation, superoxide generation and elastase secretion); (c) suppression of IL-8 production by epithelia and leukocytes; (d) upregulation of bactericidal permeability-increasing protein expression in epithelial cells; (e) upregulation of monocyte chemotaxis; (f) upregulation of monocyte ingestion of apoptotic neutrophils (Ernst Schering Res Found Workshop 31:143, 2000; Prostaglandins 53:107, 1997; Braz J Med Biol Res 34, 555, 2001; Prostaglandins & other Lipid Mediators 68-69:433, 2002; Proc Natl Acad Sci USA 99:13266, 2002; Proc Natl Acad Sci USA 99:3902, 2002). In a variety of of in vivo models, LX have been shown to prevent neutrophil-mediated damage, and promote the resolution of neutrophil-mediated inflammation (Ernst Schering Res Found Workshop 31:143, 2000; Prostaglandins 53:107,1997; Braz J Med Biol Res 34, 555, 2001; Prostaglandins & other Lipid Mediators 68-69:433, 2002). Notably, LX analogues have recently been shown to downmodulate allergic pulmonary inflammatory responses in mouse models (Nature Immunol 8:1018, 2002).

It has previously been shown that sputum LXA₄ concentrations are significantly higher in mild compared with severe asthmatics (Bonnans, C. et al. Am J Respir Crit Care Med 165, 1531, 2002), and that patients with aspirin-tolerant asthma generate more lipoxins in whole blood stimulation assays than do asthma-intolerant asthmatics (Sanak, M. et al. Eur Respir J 16, 44, 2000). Together with these previous studies, the current data suggest the likelihood that deficiencies in lipid counter-regulatory mediators such as lipoxins may well play an essential or exacerbating role in a variety of diseases marked by dysregulated inflammatory processes.

As with all pathophysiologically important abnormalities described to date in the CF lung, it remains a significant scientific challenge to elucidate the linkage between abnormalities in lipoxin metabolism and the underlying gene, CFTR (Pilewski, J. M. et al, Physiol Rev 79, S215,1999). Lipoxins are generated largely transcellularly by the sequential action of lipoxygenases in cells such as airway epithelia and neutrophils (Serhan, C. N. Ernst Schering Res Found Workshop 31, 143, 2000). Any mechanism proposed to underlie aberrant lipoxin metabolism in the CF airway needs to come to terms with the fact that the production of LTB₄ (derived from arachidonic acid, dependent upon lipoxygenase activity, catabolized by the same pathways as LXA₄) is upregulated in that environment, as is the secretion of prostaglandin (PG)E₂, an eicosanoid mediator that both inhibits LTB₄ and upregulates LXA₄ production (Konstan, M. W., et al Am Rev Respir Dis 148, 896, 1993; Levy, B. D., et al. Nat Immunol 2, 612, 2001). It should also be noted that, while the enzymatic events needed for the generation of LXA₄ are clear (that is, oxygenation at the C5 and C15 positions), the specific lipoxygenase enzymes that are critical for LXA₄ production in the lung remain to be clarified. Rigorous biochemistry remains to be done with many of these lipoxygenases.

While a primary defect in neutrophils might be envisaged, this is not consonant with the fact that aberrant neutrophilic inflammation is only seen in the airways in CF. Further, a wide variety of transcriptional and functional changes have been found downstream of CFTR mutations in airway epithelia, providing a likely locus for abnormalities in lipoxin metabolism in the CF airway (Xu, Y, et al, J Biol Chem 278, 7674, 2003). Indeed, microarray analysis of lungs from mice with pulmonary expression of human ΔF508 CFTR has revealed significant decreases in the expression of 2 lipoxygenases in the basal, unstimulated state, something that provides a rational entree into the search for the mechanistic link between CFTR mutations and abnormalities in lipoxin metabolism.

### SUMMARY OF THE INVENTION

The present invention demonstrates a pathophysiologically important defect in lipoxin-mediated anti-inflammatory activity in the CF lung, and that therapy with lipoxin analogues (or upregulation of endogenous lipoxin production) may have therapeutic potential for ameliorating pathogenic inflammatory responses in CF.

The present invention also demonstrates that lipoxin analogue treatment of P. aeruginosa-infected mice led both to inhibition of pulmonary neutrophil accumulation, as well as to better control of the infectious challenge (along with reduced morbidity). Clearly, lipoxin-mediated down-modulation of the neutrophilic response does not compromise bacterial clearance. For one, continuing, vigorous neutrophilic inflammation may not be the optimal protective response for airway infection with P. aeruginosa. Indeed, quite vigorous neutrophilic inflammation, in the absence of Pseudomonas clearance, is seen in the C5aR-deficient mouse as well as in CF patients (Hopen, U. E., et al, Nature 383, 25, 1996). Further, however, treatment with LXA₄ leads to maturation of the inflammatory response: not just down-regulation of neutrophil accumulation, but upregulation of mononuclear cell accumulation as well. This change in the character of the inflammatory response may well be responsible for more efficient bacterial clearance.

The present invention shows that treatment of primary human bronchial epithelia with nanomolar concentrations of an LXA₄ analogue significantly inhibits bacterially-driven IL-8 production by such cells. Although lipoxin research has largely focused on the immunoregulatory properties of these lipids, lipoxins have additional properties of potential relevance to CF. The physiological importance of lipoxin-driven epithelial ion transport (and defects therein) at sites of mucosal inflammation remains to be determined.

This invention provides a method for preventing or treating pulmonary disease in patients with cystic fibrosis and related diseases including variant cystic fibrosis, and non-cystic fibrosis bronchiectasis. The method involves administering a pharmaceutically effective amount of a lipoxin or a metabolically stable lipoxin analogue to patients with cystic fibrosis, in amounts sufficient to downregulate harmful neutrophilic airway inflammatory responses.

The types of LX-type compounds that can be used in the present invention are generally represented by formula 1 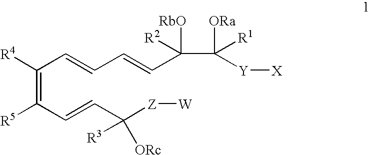
wherein:
X is selected from the group consisting of: —C(O)-A, —SO2-A, —PO(OR)-A, where A is hydroxy, alcoxy, aryloxy, amino, alkylamino, dialkylamino, or —OM, where M is a cation selected from the group consisting of ammonium, tetra-alkyl ammonium, Na, K, Mg, and Zn,
Y and Z are linkers independently selected from the group consisting of a chain of up to 20 atoms and a ring containing up to 20 atoms, provided that Y and Z can independently include one or more nitrogen, oxygen, sulfur or phosphorous atoms, and further provided that Y and Z can independently include one or more substituents selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that Y and Z can also contain one or more fused carbocyclic, heterocyclic, aryl or heteroaryl rings, and provided that linkers Y and Z are connected to the adjacent C(R)OR group via a carbon atom or a C-heteroatom bond where the heteroatom is oxygen, sulfur, phosphorous or nitrogen;
W is selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.
Ra, Rb and Rc, are independently selected from the group consisting of hydrogen, alkyl, aryl, heteroaryl, acyl, silyl, alcoxyacyl and aminoacyl;
R¹, R² and R³ are independently selected from the group consisting of hydrogen, alkyl, aryl and heteroaryl, provided that R¹, R² and R³ can independently be connected to linkers Y or Z;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond.

Among the preferred embodiments of the present invention are compounds selected from the group consisting of the following compound 2 and 3 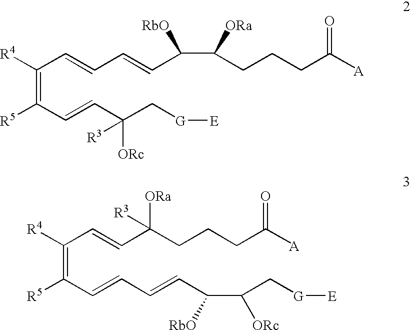
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
The stereochemistry at C-15 can be R or S.

A typical LXA4 analogue used in the present invention has the following structure 4, 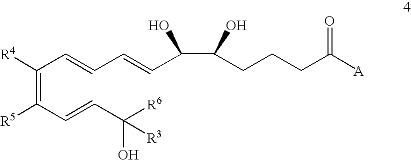
wherein:
A is selected from a group consisting of: hydroxy, methoxy, alcoxy, aryloxy, amino, alkylamino, dialkylamino, alkoxyamino, hydroxyamino,
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³ and R⁶ may be connected together to form a carbocyclic, or heterocyclic ring of 3 to 8 atoms, including rings containing O, S and N atoms; and
the stereochemistry at C-15 can be R or S.

A typical LXB4 analogue used in the present invention has the following structure 5, 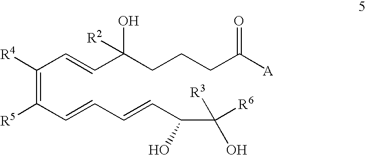
wherein:
A is selected from a group consisting of: hydroxy, methoxy, alcoxy, aryloxy, amino, alkylamino, dialkylamino, alkoxyamino, hydroxyamino,
R² and R³ are independently selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³ and R⁶ may be connected together to form a carbocyclic, or heterocyclic ring of 3 to 8 atoms, including rings containing O, S and N atoms; and
the stereochemistry at C-15 can be R or S.

It is the object of the present invention to provide compositions containing lipoxin analogues that mimic the structures and properties of natural lipoxins belonging to the LXA, 15-epi-LXA, LXB, or 15-epi-LXB series. In one embodiment, the invention provides compounds and compositions that are shown to have properties that enable them to modulate airway inflammation associated with cystic fibrosis.

In another embodiment, the invention features methods of preventing, ameliorating or treating conditions associated with airway inflammation in patients with cystic fibrosis, as well as variant cystic fibrosis, and non-cystic fibrosis bronchiectasis. The method comprises administering a lipoxin or lipoxin analogue, (suspended in a pharmaceutically acceptable carrier), in an amount sufficient to treat, prevent, or modulate neutrophilic airway inflammatory responses.

Also included is a method of administering a lipoxin or lipoxin analogue in combination with a therapeutic agent in an amount sufficient to treat prevent, or modulate neutrophilic airway inflammatory responses in subjects with CF-related disease.

### BRIEF DESCRIPTION OF THE DRAWINGS

FIG. 1 depicts ELISA results of the levels of LXA4 and IL-8 in bronchoalveolar lavage fluids from stable patients with cystic fibrosis and pulmonary inflammatory controls. In the figure, CF=cystic fibrosis; IC=inflammatory control; n=12 and 8 for CF and IC, respectively.

FIG. 2 is a graph showing Lipoxin A4 analogue inhibition of P. aeruginosa-mediated IL-8 secretion by primary human bronchial epithelial cells. In the figure, * P<0.002. The figure is representative of 4 separate experiments.

FIG. 3 is a graph showing amelioration of disease in mice challenged with mucoid P. aeruginosa entrapped in agar beads and subsequently treated with LX analogue. In the figure means±SE are shown, n=5 for controls; n=6 for mice treated with LX analogue. The figure is representative of 2 separate experiments. * P<0.05, ** P<0.01.

FIGS. 4A through 4F present data form analysis of cells in bronchoalveolar lavage from mice that were untreated (control) or treated (lipoxin) after challenged with mucoid P. aeruginosa entrapped in agar beads. In the figure means±SE are shown, filled bars=LXA₄ analogue treatment; hatched bars=vehicle control treatment, n=6 for mice treated with LXA₄ analogue; n=5 for controls. The figure is a representative of 2 separate experiments. Airway cell numbers in C57BL/6 mice of similar age who did not undergo challenge with P. aeruginosa entrapped in agarose beads (n=14): total cells=59,773±(SE) 6,811; neutrophils=15±9; lymphocytes=47±24; macrophages=32,806±5,020.

FIG. 4A is a graph depicting a decrease in total cell counts after treatment with LX analogue

FIG. 4B is a graph depicting a decrease in airway neutrophils.

FIG. 4C is a graph depicting an increase in airway lymphocyte counts

FIG. 4D is a graph depicting an increase in the percentage of airway macrophages

FIG. 4E is a graph depicting a decrease in neutrophil/lymphocyte ratio

FIG. 4F is a graph depicting a decrease in neutrophil/macrophage ratio

FIG. 5 is a graph showing a decrease in whole lung myeloperoxidase levels of mice challenged with P. aeruginosa entrapped in agar beads, after treatment with lipoxin. In the figure means±SE are shown, filled bars=LXA₄ analogue treatment; hatched bars=vehicle control treatment, n=6 for mice treated with LXA₄ analogue; n=5 for controls. The figure is representative of 2 separate experiments.

FIG. 6 is a graph showing a decrease in whole lung P. aeruginosa burden in lipoxin treated vs. untreated mice challenged with P. aeruginosa entrapped in agar beads. In the figure means±SE are shown, filled bars=LXA₄ analogue treatment; hatched bars=vehicle control treatment, n=6 for mice treated with LXA₄ analogue; n=5 for controls. The figure is representative of 2 separate experiments.

### DETAILED DESCRIPTION OF THE INVENTION

Definitions:

Unless defined otherwise, all technical and scientific terms used herein have the same meaning as is commonly understood by one of ordinary skill in the art to which the claimed subject matter belongs. All patents, applications, published applications and other publications are incorporated by reference in their entirety. In the event that there are a plurality of definitions for a term herein, those in this section prevail unless stated otherwise.

As used in this specification, alkyl groups can include straight-chained, branched and cyclic alkyl radicals containing up to about 20 carbons. Suitable alkyl groups may be saturated or unsaturated. Further, an alkyl may also be substituted one or more times on one or more carbons with substituents selected from the group consisting of C1-C6 alkyl, C3-C6 heterocycle, aryl, halo, hydroxy, amino, alkoxy and sulfonyl. Additionally, an alkyl group may contain up to 10 heteroatoms or heteroatom substituents. Suitable heteroatoms include nitrogen, oxygen, sulfur and phosphorous.

As used in this specification, aryl groups are aryl radicals which may contain up to 10 heteroatoms. An aryl group may also be optionally substituted one or more times with an aryl group or a lower alkyl group and it may be also fused to other aryl or cycloalkyl rings. Suitable aryl groups include, for example, phenyl, naphthyl, tolyl, imidazolyl, pyridyl, pyrroyl, thienyl, pyrimidyl, thiazolyl and furyl groups.

As used in this specification, a ring is defined as having up to 20 atoms that may include one or more nitrogen, oxygen, sulfur or phosphorous atoms, provided that the ring can have one or more substituents selected from the group consisting of hydrogen, alkyl, allyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that the ring may also contain one or more fused rings, including carbocyclic, heterocyclic, aryl or heteroaryl rings.

The terms “subject” and “patient” mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest. “Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing or eradicating a deleterious or harmful condition associated with CF-related disease. Examples of such treatment include: decreasing bacterial infection, increasing pulmonary function, down regulation of pro-inflammatory cytokines and upregulating mononuclear cell accumulation.

For the purposes of this application, the terms “CF-related disease(s) or disorder(s)” includes diseases and/or conditions related to Cystic Fibrosis (CF). Examples of such diseases include cystic fibrosis, variant cystic fibrosis and non-CF bronchiectasis.

“Cystic fibrosis (CF)” is an autosomal recessive disorder with a highly variable clinical presentation. Cystic fibrosis is predominantly a disorder of infants, children and young adults, in which there is widespread dysfunction of the exocrine glands, characterized by signs of chronic pulmonary disease, pancreatic deficiency, abnormally high levels of electrolytes in the sweat and occasionally by biliary cirrhosis. Also associated with the disorder is an ineffective immunologic defense against bacteria as well as dysregulated inflammation in the lungs. The classic form of cystic fibrosis is caused by loss-of-function mutations in the cystic fibrosis transmembrane conductance regulator (CFTR) gene. Nonclassic forms of cystic fibrosis have been associated with mutations that reduce but do not eliminate the function of the CFTR protein.

“Variant cystic fibrosis” is a disorder which is phenotypically indistinguishable from cystic fibrosis, but which is not associated with mutations in the CFTR gene (N Engl J Med. 2002; 347: 401-7).

“Bronchiectasis” is primarily the result of airway injury and remodeling attributable to recurrent or chronic inflammation and infection. The underlying etiologies include autoimmune diseases, severe infections, genetic abnormalities, and acquired disorders. Recurrent airway inflammation and infection may also be the result of allergic or immunodeficiency states such as allergic bronchopulmonary mycoses or HIV/AIDS. Bronchiectasis is included in the differentiation diagnosis of any patient with chronic respiratory complaints such as cough and sputum production. Early clinical manifestations may be subtle. Hallmarks of severe bronchiectasis include fetid breath, chronic cough, and sputum production. The associated chronic respiratory infections and airway sepsis are punctuated by episodes of acute exacerbation.

“Modulating airway inflammation” as used herein refers to a change in airway inflammatory process associated with CF-related diseases or disorders. Methods of treating, preventing and modulating neutrophilic airway inflammatory responses are well known in the art and can include (a) causing downregulation of neutrophil chemotactic mediators such as IL-8; (b) causing a decrease in the accumulation of neutrophils in the airways; (c) causing suppression or prevention of airway neutrophil activation; and (d) upregulating mononuclear cell infiltration into the airway.

A “therapeutic agent”, as used herein, may be any compound, chemical or biological substance that will have a beneficial effect when administered to such a subject. Examples of therapeutic agents useful in the practice of the invention include anti-inflammatory substances (e.g. corticosteroids, NSAIDS or ibuprofen), antimicrobial substances, bronchodilators, mucolytic agents, DNAse or ion-channel modulating agent.

Lipoxins and Lipoxin Analogues

There are a number of isomeric lipoxins that are derived from arachidonic acid or eicosapentaenoic acid, including those shown in Scheme 1. For the purposes of the present invention, a lipoxin analogue (LX analog) is a molecule that has a structure similar to the structure of a lipoxin (LX) selected from Scheme 1, whereby a portion of the selected LX molecule has been replaced with a structurally equivalent moiety.

It is the object of the present invention to provide compositions containing lipoxin analogues that mimic the structures and properties of natural lipoxins belonging to the LXA or LXB series. In one embodiment, the invention provides compounds and compositions that are shown to have properties that enable them to modulate airway inflammation associated with cystic fibrosis.

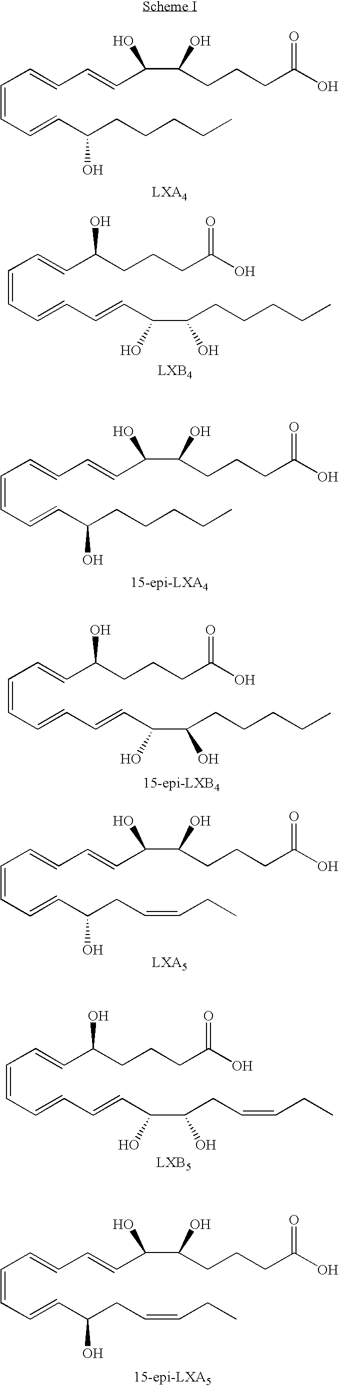

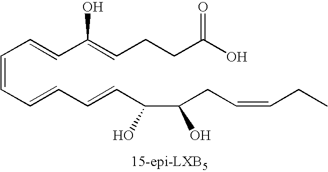

Examples of suitable lipoxin analogues include, but are not limited to the following:

Compounds of the general formula 1 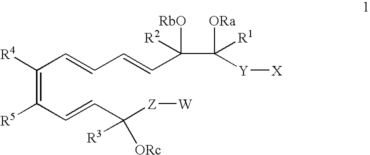
wherein:
X is selected from the group consisting of: —C(O)-A, —SO₂-A, —PO(OR)-A, where A is hydroxy, alcoxy, aryloxy, amino, alkylamino, dialkylamino, or —OM, where M is a cation selected from the group consisting of ammonium, tetra-alkyl ammonium, Na, K, Mg, and Zn,
Y and Z are linkers independently selected from the group consisting of a chain of up to 20 atoms and a ring containing up to 20 atoms, provided that Y and Z can independently include one or more nitrogen, oxygen, sulfur or phosphorous atoms, and further provided that Y and Z can independently include one or more substituents selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that Y and Z can also contain one or more fused carbocyclic, heterocyclic, aryl or heteroaryl rings, and provided that linkers Y and Z are connected to the adjacent C(R)OR group via a carbon atom or a C-heteroatom bond where the heteroatom is oxygen, sulfur, phosphorous or nitrogen;
W is selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.
Ra, Rb and Rc, are independently selected from the group consisting of hydrogen, alkyl, aryl, heteroaryl, acyl, silyl, alcoxyacyl and aminoacyl;
R¹, R² and R³ are independently selected from the group consisting of hydrogen, alkyl, aryl and heteroaryl, provided that R¹, R² and R³can independently be connected to linkers Y or Z;
R⁴ and R⁵ are independently selected from the group consistirig of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond.

Among the preferred embodiments of the present invention are compounds selected from the group consisting of the following compound 2 or 3 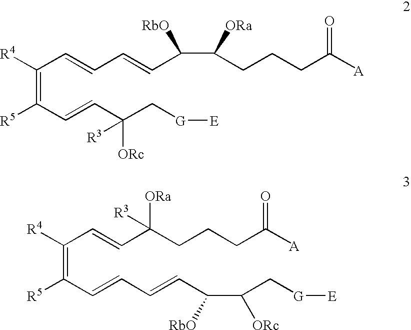
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
The stereochemistry at C-15 can be R or S.

In other embodiments, the composition provided herein contain a lipoxin analogue that has the following structure 6 or 7, 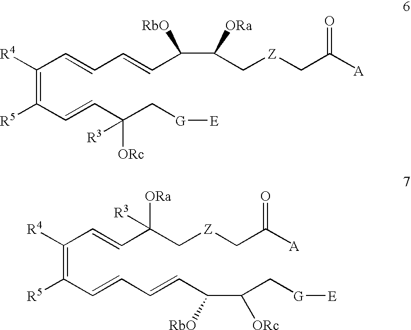
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Z is independently selected from the group consisting of: —O—, —S(O)—, —S(O)₂—, —C(═O)—, —N(Rd)-, —N(CORd)-, —N(SORd)-, —N(SO₂Rd)-, where Rd is alkyl, aryl, alcoxy, amido or —(CXY)—, where X and Y are hydrogen, halo, hydroxy, alcoxy, amino, amido, sulfonamido, alkyl, aryl, or acyl, and provided that X and Y can be joined together to form a carbocyclic or heterocyclic ring;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
the stereochemistry at C-15 can be R or S.

A typical LXA4 analogue used in the present invention has the following structure 4, 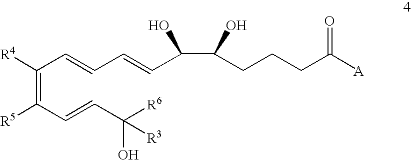
wherein:
A is selected from a group consisting of: hydroxy, methoxy, alcoxy, aryloxy, amino, alkylamino, dialkylamino, alkoxyamino, hydroxyamino,
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³ and R⁶ may be connected together to form a carbocyclic, or heterocyclic ring of 3 to 8 atoms, including rings containing O, S and N atoms; and
the stereochemistry at C-15 can be R or S.

A typical LXB4 analogue used in the present invention has the following structure 5, 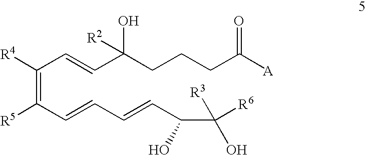
wherein:
A is selected from a group consisting of: hydroxy, methoxy, alcoxy, aryloxy, amino, alkylamino, dialkylamino, alkoxyamino, hydroxyamino,
R² and R³ are independently selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³ and R⁶ may be connected together to form a carbocyclic, or heterocyclic ring of 3 to 8 atoms, including rings containing O, S and N atoms; and
the stereochemistry at C-15 can be R or S.
   Preparation of Lipoxins and Lipoxin Analogues

The synthesis of lipoxins and lipoxin analogues can be performed using methods known to those skilled in the art (Angew. Chem. Int. Ed. Engl., 30:1100, 1991). A typical synthesis involves the preparation of certain key intermediates, which are finally coupled together to form the final molecule. A typical synthesis is outlined in Scheme 2. 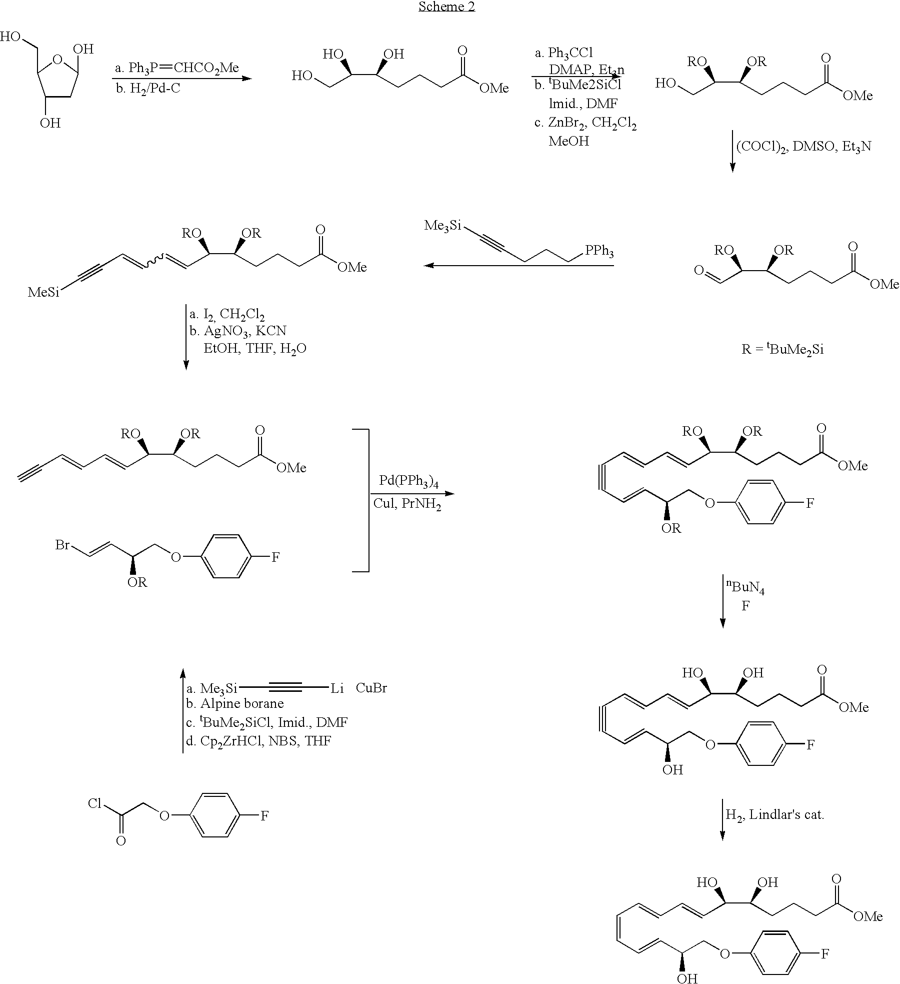
Lipoxins and Lipoxin Analogues in Cystic Fibrosis:

In another embodiment, the invention features methods of preventing, ameliorating or treating conditions associated with airway inflammation in patients with cystic fibrosis, as well as variant cystic fibrosis, and non-cystic fibrosis bronchiectasis. The method comprises administering a lipoxin or lipoxin analogue (suspended in a pharmaceutically acceptable carrier), in an amount sufficient to treat, prevent, or modulate neutrophilic airway inflammatory responses

In the present invention we have discovered the previously unknown potential therapeutic role of the lipoxins and stable lipoxin analogues in cystic fibrosis. We have established for the first time that: (1) the LX-associated activities, as well as LX, are deficient in the dysregulated inflammatory environment of the CF lung; and (2) administration of a metabolically stable LX analogue in a well characterized mouse model of the chronic airway inflammation and infection of cystic fibrosis has beneficial therapeutic effects, including: (a) suppression of neutrophilic inflammation; (b) a shift towards chronic inflammation; (c) decreased lung bacterial burdens; and (d) attenuated disease severity, marked by less loss of weight, along with earlier and more complete recovery thereof.

Based on the identification of an LX deficiency associated with the dysregulated airway neutrophilic inflammation in CF, the present invention establishes a new method for CF treatment involving LX and LX analogs.

The molecular mechanisms responsible for LX deficiency in CF remain unclear. Potentially relevant alterations in lipid metabolism have been described in CF, however. Fatty acid deficiencies are well described in CF. Deficiencies in essential fatty acids (including omega-3 fatty acids [\tilde{ω}3FA]) in patients with CF are not due to malnutrition (Eur J Pediatr 156:952, 1997). On the other hand, AA levels are elevated in the phospholipid fraction of BALF from patients with CF, compared not only to healthy controls, but also to patients with chronic bronchitis, and non-CF patients with P. aeruginosa pneumonia (Scand J Clin Lab Invest 46:511, 1986). Both of these observations suggest that lipid abnormalities may be a fairly direct result of mutations in CFTR. Long term intravenous supplementation with essential fatty acids appears to ameliorate the course of disease in CF; the mechanism has remained obscure and the treatment itself is impractical. Further, CFTR-/- mice exhibit a marked abnormality in the ratio of phospholipid-bound AA (up) to docosahexaenoic acid (DHA; C22:6; an \tilde{ω} 3FA) [down] (Proc Natl Acad Sci USA 96:13995, 1999). Notably, significant deviation from normal AA/DHA ratios is predominantly seen in tissues that express CFTR. Overfeeding with DHS normalized this ratio in these tissues, and inhibits the airway inflammatory response to endotoxin, in CFTR-/- mice (Proc Natl Acad Sci USA 96:13995, 1999; J Appl Physiol 92:2169, 2002). The mechanism is unknown. Similarly, the mechanism underlying the ability of therapy with high dose ibuprofen to spare lung function in children with CF remains unclear. Notably, however, both effects may well be due to the generation of LX and related molecules. Thus, it has recently been reported that novel sets of anti-inflammatory LX-related mediators are generated from ω 3FA, particularly in the presence of COX-2 inhibition with nonsteroidal antiinflammatory drugs (J Exp Med 192:1197, 2000; J Exp Med 196:1025, 2002; J Biol Chem 278:14677, 2003).

Therefore, we propose that there is a pathophysiologically-important defect in LX-mediated antiinflammatory activity in the airways of pateints with CF, as well as in the airways of patients with variant CF and non-CF bronchiectasis. The suppressed levels of LX in CF, first documented herein, are consistent with previous reports that the transcription of 15-lipoxygenase and COX-2 is suppressed or diminished in CF (Thorax, 51:1223, 1996). Since these enzymes are involved in the formation of lipoxins, their diminished levels in CF may explain why LX-mediated resolution of neutrophilic pulmonary inflammation is also suppressed. Furthermore, the documented imbalance in membrane lipids in CF (Proc Natl Acad Sci USA, 96:13995, 1999) may exacerbate the situation due to the additional suppression of alternative anti-inflammatory lipids that can be generated from DHA and other polyunsaturated fatty acids. Overall, our findings are consistent with a major imbalance between anti-inflammatory and pro-inflammatory lipid mediators in CF. Therefore, according to the present invention, the administration of anti-inflammatory lipoxin or lipoxin analogues can correct this imbalance.

The present invention provides a method for treating pulmonary disease in patients with CF, variant CF, and non-cystic fibrosis bronchiectasis. The method comprises administering an LX or an LX analogue (suspended in a pharmaceutically acceptable carrier), in an amount sufficient to treat, prevent, or modulate neutrophilic airway inflammatory responses, to patients with CF, patients with variant CF, and patients with non-cystic fibrosis bronchiectasis.

Pharmaceutical Compositions

The compounds of the invention can be incorporated into pharmaceutical compositions suitable for administration to a subject. Such compositions typically contain the active compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the treatment methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Depending on the subject and condition being treated and on the administration route, the subject compounds may be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release

The pharmaceutical compositions can be included in a container, pack, or dispenser together with instructions for administration.

Therapeutic Uses

The invention provides compounds that are shown to have properties that enable them to modulate airway inflammation associated with cystic fibrosis. Accordingly, in one aspect the invention features methods of preventing, ameliorating or treating conditions associated with airway inflammation in patients with cystic fibrosis, as well as variant cystic fibrosis, and non-cystic fibrosis bronchiectasis. The method comprises administering a lipoxin or lipoxin analogue (suspended in a pharmaceutically acceptable carrier), in an amount sufficient to treat, prevent, or modulate neutrophilic airway inflammatory responses.

Identification of a subject with CF-related disease can be performed by standard methods in the art. Examples include genetic testing, sweat tests, and tests for abnormal CF transmembrane conductance regulator (CFTR) protein function. After administering a lipoxin or lipoxin analogue in an amount sufficient to treat, prevent, or modulate neutrophilic airway inflammatory response a subject may be monitored to determine the efficaciousness of the administration. For example, subjects may be monitored for: (a) decreased bacterial infection, (b) increased pulmonary function or decreased decrement in pulmonary function over time, (c) decreased levels of airway neutrophils or products of airway neutrophils; (d) decreased levels of airway pro-inflammatory cytokines or proinflammatory lipid mediators; (e) recovery of weight loss.

Also included is a method of administering a lipoxin or lipoxin analogue, in combination with a therapeutic agent in an amount sufficient to treat prevent, or modulate neutrophilic airway inflammatory responses in subjects with CF-related disease. A “therapeutic agent”, as used herein, may be any compound, chemical or biological substance that will have a beneficial effect when administered to such a subject. Examples of therapeutic agents useful in the practice of the invention include anti-inflammatory substances (e.g. corticosteroids, NSAIDS or ibuprofen), antimicrobial substances, bronchodilators, mucolytic agents, DNAse or ion-channel modulating agent.

The invention will be further described in the following examples, which are illustrative only, and which are not intended to limit the scope of the invention described in the claims.

### EXAMPLES

In the following examples, efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric. Reagents and materials used in these examples are generally either commercially available or can be readily prepared from commercially available reagents by a procedure involving one or more steps.

### Example 1

### Preparation of 11-dehydro-15-epi-16-(p-fluorophenoxy) LXA4 methyl ester

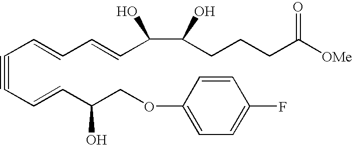

Part 1: Ooxalyl chloride (0.47 mL, 5.4 mmol) was added dropwise at −78° C. to a solution of dimethyl sulfoxide (0.56 mL, 7.20 mmol) in CH₂Cl₂ (9 mL) and the solution was stirred at that temperature for 15 min. (5S, 6R) Methyl 5,6-di(tert-butyldimethylsiloxy)-7-hydroxyheptanoate (1,516 mg, 3.60 mmol) in CH₂Cl₂ (9 mL) was added with the help of a double-end needle and the solution was stirred an additional 45 min. at −78° C. At this point, triethylamine (2.5 mL, 18 mmol) was added slowly to the clowdy white mixture, resulting in the formation of a white solid. The mixture was allowed to reach 25° C. and it was then poured into water and extracted with ethyl acetate. The combined extracts were washed with brine, dried over Na₂SO₄ and the solvent was evaporated. Flash column chromatography (silica, 5% ethyl acetate/hexanes) afforded pure (5S, 6R) methyl 5,6-di(tert-butyldimethylsiloxy)-7-oxoheptanoate as a colorless liquid (1,353 mg, 89% yield). ¹H NMR (500 MHz, C₆D₆): δ 9.60 (d, J=1.4 Hz, 1H), 3.90 (m, 2H), 3.37 (s, 3H), 2.10 (t, J=6.9 Hz, 2H), 1.60 (m, 4H), 0.94 (s, 18H), 0.08 (s, 3H), 0.05 (s, 6H), 0.03 (s, 3H). ¹³C NMR (500 MHz, C₆D₆): δ 201.44, 172.45, 80.74, 74.82, 50.55, 33.43, 32.82, 25.63, 25.56, 20.47, 18.03, 17.89, −4.74, −4.96, −5.00, −5.29.

Part 2: To a solution of 5-trimethylsilyl-2-pentene-4-yn-1-ol (2,470 mg, 16.00 mmol) and triphenylphosphine (5,090 mg, 19.21 mmol) in CH₂Cl₂ (32 mL) cooled at 0° C. was added slowly N-bromosuccinimide (3,166 mg, 17.61 mmol). The reaction mixture was brought to 25° C. and stirred for 20 min. It was then quenched with saturated aqueous solution of sodium bicarbonate. The product was extracted with ether, washed with brine, dried over Na₂SO₄ and the solvent was evaporated. Flash column chromatography (silica, 4% ether/hexanes) afforded the pure product as a colorless liquid (2,803 mg, 73% overall from 2-pentene-4-yn-1-ol). ¹H NMR (500 MHz, CDCl₃) δ 6.27 (dt, J=15.6 Hz, J=7.9 Hz, 1H), 5.73 (d, 15.63.94 (dd, J=7.9 Hz, J=1.1 Hz, 2H), 0.14 (s, 9H). A solution of this bromide (1,833 mg, 8.44 mmol) and triphenylphosphine (2,683 mg, 10.13 mmol) in benzene (16 mL) were stirred for 2 days. The whitish solid phosphonium salt product was filtered, washed three times with ether and dried under vacuo next to P₂O₅.

Part 3: A solution of dried phosphonium salt from Part 2 (1589 mg, 3.31 mmol) in THF (20 mL) was treated with n-BuLi (1.2 mL of 2.5M solution in hexanes, 0.93 mmol) at −78° C. The brown solution was allowed to warm to 0° C. and recool at −78° C. at which point a solution of (5S, 6R) methyl 5,6-di(tert-butyldimethylsiloxy)-7-oxoheptanoate from Part 1 (980 mg, 2.08 mmol) in THF (10 mL) was added dropwise. The reaction mixture was brought to 25° C. and stirred for 2 h. It was then quenched with saturated aqueous solution of ammonium chloride. The product was extracted with ether, washed with brine, dried over Na₂SO₄ and the solvent was evaporated. Flash column chromatography (silica, 8% ether/hexanes) afforded (5S,6R) methyl 5,6-di(tert-butyldimethylsiloxy)-12-trimethylsilyl-dodeca-7,9-diene-11-yn-noate as a mixture of E,E and E,Z isomers (1,121 mg, 100% yield). A solution of this product (1,121 mg, 2.08 mmol) and a small piece of iodine (calcd 5 mg) in CH₂Cl₂ (20 mL) was strirred at 25° C. for 24 h. It was then quenched with saturated aqueous solution of sodium metabisulfite. The product was extracted with ether, washed with brine, dried over Na₂SO₄ and the solvent was evaporated. Flash column chromatography (silica, 3% ether/hexanes) afforded pure (7E, 9E, 5S, 6R) methyl 5,6-di(tert-butyldimethylsiloxy)-12-trimethylsilyl-dodeca-7,9-diene-11-yn-noate as a colorless liquid (910 mg, 81% yield). A solution of this compound (420 mg, 0.78 mmol) in THF/EtOH (2 mL/1 mL) was treated with silver nitrate (529 mg, 3.11 mmol) in H₂O/EtOH (2 mL/1 mL) at 0° C. The resulting yellow solid suspension was allowed to warm to 25° C. and it was then treated with potasium cyanide (355 mg, 5.45 mmol) in H₂O (1 mL). The product was extracted with ether, washed with brine, dried over Na₂SO₄ and the solvent was evaporated. Flash column chromatography (silica, 3% ether/hexanes) afforded (7E, 9E, 5S, 6R) methyl 5,6-di(tert-butyldimethylsiloxy)-dodeca-7,9-diene-11-yn-noate as a pure colorless liquid (703 mg, 89% yield). ¹H NMR (500 MHz, CDCl₃): δ 6.32 (dd, J=15.6 Hz and 6.5 Hz, 1H), 6.14 (dd, J=15.2 Hz and 10.9 Hz, 1H), 5.75 (dd, J=15.2 Hz and 7.1 Hz, 1H) 5.52 (d, J=15.8 Hz, 1H), 3.98-3.95 (m, 1H), 3.64 (s, 3H), 3.56-3.53 (m, 1H), 3.00 (s, 1H), 2.27 (t, J=7.4 Hz, 2H), 1.71-1.41 (m, 4H), 0.86 (s, 9H), 0.84 (s, 9H), 0.02 (s, 3H), 0.01 (s, 3H), −0.01 (s, 3H), −0.02 (s, 3H). ¹³C NMR (500 MHz, CDCl₃): δ 172.88, 139.11, 134.54, 128.31, 115.30, 83.82, 81.11, 77.98, 74.14, 50.54, 33.82, 32.42, 25.87, 25.64, 20.18, 18.22, 17.41, −4.11, −4.30, −4.83, −5.08.

Part 4. Copper bromide-dimethylsulfide complex (2.16 g, 10.15 mmol) was suspended in ether (30 mL) and treated with a freshly prepared etheral solution of lithium trimethylsilyl-acetylide (20.3 mmol) at −78° C. The mixture was brought to −20° C., at which point it turned clear, and then was cooled to −78° C. (4-Fluorophenoxy) acetyl chloride (20.3 mmol) was added to the solution, the reaction mixture was brought to room temperature and stirred for 1 hr. It was then quenched with saturated solution of ammonium chloride, washed with brine, dried and concentrated. Flash chromatography (silica gel, 5% ether/hexanes) gave 1-trimethylsilyl-4-(4-fluorophenoxy)-1-butyn-3-one as a colorless liquid (71% yield). This ketone (2.0 g, 12.5 mmol) was dissolved in 2 mL of THF, cooled to 0° C., and R-alpine borane (4.84 g, 18.75 mmol) was added. The reaction mixture was kept at 0° C. for 24 h, after which time it was treated with freshly distilled acetaldehyde. The liberated pinene was removed at 80° C. under reduced pressure, and the residue was slowly treated with NaOH/H₂O₂ It was then extracted with ether, dried and concentrated. The crude material was chromatographed (silica gel, 15% ethyl acetate/hexanes) to give (S)-4-(4-fluorophenoxy)-1-trimethylsilylbutyne-3-ol as a colorless liquid (88% yield, 94% ee). To a solution of this compound (4.28 mmol) in CH₂Cl₂ (10 ml) was added 2,6-lutidine (1.12 ml, 9.4 mmol) and tert-butyldimethylsilyloxy triflate (1.47 ml, 6.42 mmol) at 0° C. The solution was warmed to room temperature and stirred for 4 hours. The resulting yellowish reaction mixture was poured into a solution of saturated NH₄Cl, extracted with ether, washed with brine, dried and concentrated. Flash column chromatography (silica gel, hexanes) afforded pure (S)-4-(4-fluorophenoxy)-3-[(tert-butyldimethylsilyl)oxy]-1-trimethylsilylbutyne as a colorless liquid in 92% yield. This compound (0.78 mmol) in THF/EtOH (2 mL/1 mL) was treated with silver nitrate (529 mg, 3.11 mmol) in H₂O/EtOH (2 mL/1 mL) at 0° C. The resulting yellow solid suspension was allowed to warm to 25° C. and it was then treated with potasium cyanide (355 mg, 5.45 mmol) in H₂O (1 mL). The product was extracted with ether, washed with brine, dried and concentrated. Flash column chromatography (silica gel, 3% ether/hexanes) afforded the pure (S)-4-(4-fluorophenoxy)-3-[(tert-butyldimethylsilyl)oxy]-1-butyne as a colorless liquid in 89% yield). To a suspension of Cp₂Zr(H)Cl (1.05 g, 3.80 mmol) in THF (2 ml) at room temperature was added a solution of (S)-4-(4-fluorophenoxy)-3-[(tert-butyldimethylsilyl)oxy]-1-butyne (3.75 mmol) in THF (2 ml) though a double tipped needle. The reaction was then protected from light and stirred for 1 h at which time it turned to a clear orange solution. At this point N-bromosuccinimide (0.68 g, 3.80 mmol) was slowly added, producing a clear yellow solution. The reaction mixture was then poured into an aqueous solution of sodium bicarbonate and extracted with ether. The combined extract were washed with brine, dried and concentrated. Flash column chromatography (silica gel, hexanes) afforded pure (3S, 1E)-4-(4-fluorophenoxy)-3-[(tert-butyldimethylsilyl)oxy]-1-bromo-1-butene as a colorless liquid in 94% yield.

Part 5. To a solution of the bromide product of Part 4 (0.32 mmol) in benzene (1 ml) was added n-propyl amine (0.13 ml, 2.1 mmol) and Pd(PPh₃)₄ (15 mg, 0.013 mmol). The solution was then protected from light and stirred at 25° C. for 45 minutes. At this point a solution of the alkyne from Part 3 (148 mg, 0.32 mmol) in benzene (1 ml) was added trough a double tipped needle followed by the addition of CuI (10 mg, 0.051 mmol). The reaction mixture was stirred for 3 h at 25° C. and then quenched with a saturated aqueous solution of ammonium chloride and extracted with ether. It was then washed with brine, dried and concentrated. Flash column chromatography (silica gel, 3% ethyl acetate/hexanes) afforded the pure (5S, 6R, 15S)-tris-(tert-butyldimethylsilyloxy)-16-(4-fluorophenoxy)-hexadeca-(7E, 9E, 13E)-trien-11-ynoic acid methyl ester as a colorless liquid in 85% yield. To a solution of this product (0.103 mmol) in THF (1 ml) cooled at 0° C. was added a 1.0 M solution of TBAF in THF (0.37 ml, 0.37 mmol) and the reaction mixture was stirred at that temperature for 3 hours. It was then poured into water and extracted with ether. The ether extracts were washed with brine, dried and concentrated affording the corresponding lactone, free acid and methyl ester. The crude solution in ether was then treated with a freshly prepared solution of diazomethane in ether to convert the free acid to the methyl ester. After removal of the ether the mixture was then dissolved in MeOH and treated with a catalytic amount of triethylamine to convert the lactone to the methyl ester product. Finally, flash column chromatography (silica gel, 4% MeOH/CH₂Cl₂) afforded pure (5S, 6R, 15S)-trihydroxy-16-(4-fluorophenoxy)-hexadeca-(7E, 9E, 13E)-trien-11-ynoic acid methyl ester or 11-dehydro-15-epi-16-(p-fluorophenoxy) LXA4 methyl ester, in 76% yield. ¹H NMR (500 MHz, C₆D₆): δ 6.77 (m, 2H), 6.64 (m, 3H), 6.22 (dd, J=15.6 Hz and 10.3 Hz, 1H), 6.11 (dd, J=15.6 Hz and 6.8 Hz, 1H) 6.04 (m, 1H), 5.73 (m, 2H), 4.32 (m, 1H), 3.95 (m, 1H), 3.51 (m, 3H), 3.41 (s, 3H), 2.12 (t, J=7.3 Hz, 2H), 1.75 (m, 1H), 1.53 (m, 1H), 1.36-1.12 (m, 2H). ¹³C NMR (500 MHz, C₆D₆): δ 174.16, 158.78, 154.96, 141.47, 141.10, 137.79, 131.27, 115.95, 112.10, 111.95, 90.87, 90.71, 75.40, 74.19, 72.18, 70.47, 51.34, 33.82, 31.49, 21.51.

### Example 2

### Preparation of 15-epi-16-(p-fluorophenoxy) LXA4 methyl ester

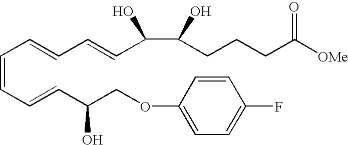

To a solution of 11-dehydro-15-epi-16-(p-fluorophenoxy) LXA4 methyl ester from Example 1 (0.04 mmol) in CH₂Cl₂ (10 ml) was added Pd-Lindlar catalyst (1.5 mg, 10% by weight), quinoline (15 μl), and the reaction mixture was stirred under the static atmosphere of hydrogen. Samples were collected every 20 minutes and analyzed by HPLC (reverse phase, 25% water/methanol) and the reaction was stopped at 60-70% conversion. The solution was then filtrated over a pad of celite to remove the catalyst, concentrated and the residue was dissolved in MeOH/water and subjected to preparative HPLC chromatography (40% water/MeOH). Finally, removal of the solvent afforded pure (5S, 6R, 15S)-trihydroxy-16-(4-fluorophenoxy)-hexadeca-(7E, 9E, 11Z,13E)-tetraenoic acid methyl ester or 15-epi-16-(p-fluorophenoxy) LXA4 methyl ester, in 50% yield. ¹H NMR (500 MHz, C₆D₆): δ 6.91 (dd, J=14.4 Hz and 9.7 Hz, 1H), 6.81 (m, 2H), 6.66 (dd, J=14.4 Hz and 10.2 Hz, 1H), 6.64 (m, 2H), 6.32 (dd, J=15.2 Hz and 10.7 Hz, 1H), 6.20 (dd, J=14.3 Hz and 10.7 Hz, 1H), 5.99 (m, 2H), 5.73 (dd, J=15.3 Hz and 6.9 Hz, 1H), 5.64 (dd, J=15.2 Hz and 6.9 Hz, 1H), 4.44 (m, 1H), 4.03 (m, 1H), 3.66-3.61 (m, 2H), 3.55 (m, 1H), 3.40 (s, 3H), 2.16 (t, J=9.2 Hz, 2H), 1.78 (m, 1H), 1.57 (m, 1H), 1.34 (m, 2H). ¹³C NMR (500 MHz, C₆D₆): δ 174.20, 158.77, 156.88, 133.82, 133.01, 132.84, 132.73, 130.38, 129.29, 128.57, 127.33, 116.04, 115.98, 75.73, 74.31, 72.76, 70.77, 51.34, 33.88, 31.60, 21.59.

### Example 3

### LX Levels are Suppressed in Bronchoalveolar Lavage Fluid

LX levels were significantly suppressed in bronchoalveolar lavage fluid (BALF) from patients with CF compared with appropriate pulmonary inflammatory controls. A study examining LXA4 levels in the CF airway was performed using BALF samples from 20 subjects.

Bronchoalveolar lavage (BAL) samples were obtained as previously described (Khan, T. Z. et al. Am J Respir Crit Care Med 151, 1075-1082 (1995).) from 20 new pediatric subjects: 12 with CF, 8 with a variety of other pulmonary inflammatory conditions, including pneumonia, interstitial lung disease, and reactive airways disease. Subjects included males and females, from ½ to 19 years of age, receiving diagnostic bronchoscopy and bronchoalveolar lavage. There were no significant differences in age or sex between patient groups.

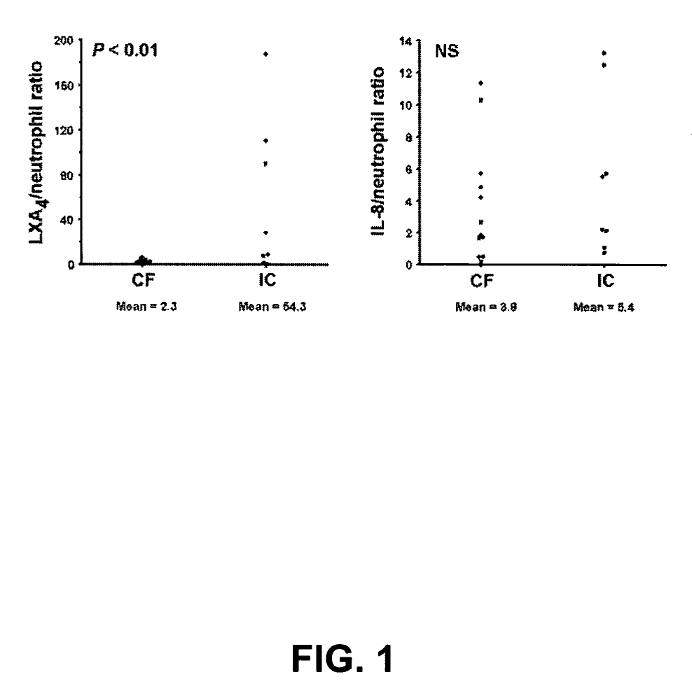

IL-8 and LXA₄ concentrations were measured by ELISA (from R&D and Oxford Biomedical Res., respectively). Informed consent was obtained from all subjects (or their parents). In particular, the following samples were examined:12 with CF, 8 with other pulmonary inflammatory conditions (including pneumonia, interstitial lung disease, and reactive airways disease). There were no statistically significant differences in neutrophil concentrations between CF patients and controls. BALF LXA4 and IL-8 concentrations were measured by ELISA (Oxford Biomedical, Oxford, Mich.) and are presented in comparison to neutrophil concentrations in FIG. 1. Significant suppression of the LXA4/neutrophil ratio was seen in patients with CF compared with inflammatory controls, in the face of comparable IL 8/neturophil ratios. The levels of LXA4 in the CF airway thus appear to be markedly suppressed for the degree of neutrophil accumulation.

### Example 4

### Human Bronchial Epithelial Cells have LX Receptors and Respond to LX Analogues

It has previously been shown that LX analogues can inhibit S. typmurium-induced secretion of IL-8 from monolayers of immortalized intestinal epithelial cells (J Immunol 167, 2772, 2001; J Clin Invest 101, 1860, 1998; J Clin Invest 92, 75, 1993). To examine the relevance of this work for airway epithelial cells, we examined whether such cells express receptors for LXA4: the high affinity LXA4 receptor (ALXR) and the CysLT1 receptor.

RNA was harvested from cultures of primary human bronchial epithelial cells (Clonetics/Cambrex), and cDNA was synthesized using Superscript II reverse transcriptase (GIBCO/Invitrogen). PCR primers were designed to amplify the entire coding region of FPRL1, the gene encoding the LXA4 receptor (Gronert, K., etal, J Exp Med 187, 1285,1998). A fragment of the anticipated size was amplified and cloned into pCR2.1 (Invitrogen). 720 bp (of a total 1.1 kb insert) was sequenced and found to be 100% identical with FPRL1. Similar PCR-mediated cloning of CYSLTR1 was done from the same cDNA using previously published primers (Gronert, K., etal, Am J Pathol 158, 3, 2001). Full nucleotide sequencing revealed 100% identity with the previously published sequence (Sarau, H. M. et al. Mol Pharmacol 56, 657, 1999; Lynch, K. R. et al. Nature 399, 789, 1999).

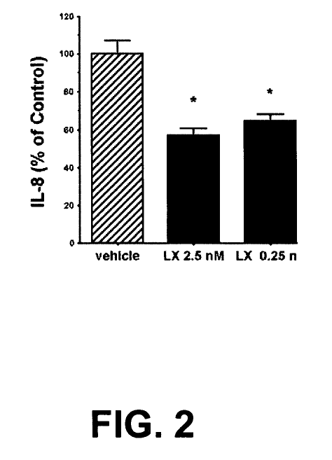

We further examined whether LX analogues were able to inhibit P. aeruginosa-induced IL 8 production from primary human bronchial epithelial cells. Primary bronchial epithelial cells from non-smokers obtained from Clonetics/Cambrex were grown until subconfluent, treated for 2 h with LXA₄ analogue or vehicle control (0.01% ethanol), stimulated for 1 h with 3×10⁷ cfu of P. aeruginosa strain PA01, and subsequently washed. Cell-free supernatants were harvested after 4 more hours of incubation, and analyzed by ELISA for IL-8 secretion. For this purpose we used the metabolically stable analog 15-epi-16-(p-fluorophenoxy) LXA4 methyl ester, prepared according to Example 2. The results are shown in FIG. 2, which shows that LX analogue 15-epi-16-(p-fluorophenoxy) LXA4 methyl ester inhibits P. aeruginosa-mediated IL-8 secretion by primary human bronchial epithelial cells. Primary human bronchial epithelial cells were pretreated with an LXA4 analogue (15-epi-16-[p-fluorophenoxy LXA₄] methyl ester) or vehicle control and subsequently stimulated with P. aeruginosa strain PA01. IL-8 was measured in cell-free supernatants. Means ±SE are shown in FIG. 2. Mean IL-8 production was 1620±116 pg/ml in vehicle control cultures.

### Example 5

### LX Analogues Modulate Inflammation in a CF Mouse Model

Administration of LX analogue 15-epi-16-(p-fluorophenoxy) LXA4 methyl ester in a well-characterized mouse model of the chronic airway inflammation and infection of cystic fibrosis was shown to have potential therapeutic benefit for CF.

To examine whether LX analogues can actually inhibit bacterial product-driven neutrophil accumulation in the airway in vivo, we turned to a well-characterized mouse model of the chronic airway inflammation and infection seen in patients with cystic fibrosis. Chronic infections with mucoid P. aeruginosa tend to persist in patients with CF. In turn, chronic airway infection with mucoid P. aeruginosa is a major cause of morbidity and mortality among CF patients. In order to achieve chronic airway infections with P. aeruginosa in murine models, it is necessary to enmesh the bacteria with an immobilizing agent such as agar (Pediatr Pulmonol 30:413.; Lab Animals 36:291). This also provides an experimental mimic of the growth of P. aeruginosa as a biofilm, which appears to be its predominant state in the CF airway (Pediatr Pulmonol 30:413, 2000; Pediatr Res 22:698, 1987; Trends Microbiol 9:50, 2001). C57/B16 mice are used, as the inflammatory response to P. aeruginosa infection of this moderately susceptible strain is a fair mimic of that seen in CF patients (Pediatr Pulmonol 30:413). Indeed, in C57/B16 mice, agar bead delivery of P. aeruginosa leads to histopathological changes, including bronchiectasis, similar to those found in patients with CF (Pediatr Pulmonol 30:413, 2000). This chronic model allows for the characterization of the effects of LX analogues on chronic, ongoing airway infection and inflammation using a model that is highly relevant to, and a reasonable mimic of, the CF airway.

Modifications were made in a previously described model (van Heeckeren, A. M. etal Lab Anim 36, 291, 2002). 6-8 week old female C57BL/6 mice were immunized in the dermis of the ear with heat-killed P. aeruginosa (mucoid strain M57-15) in incomplete Freund's adjuvant 10 days prior to P. aeruginosalagar bead challenge. Mice were challenged intratracheally (non-traumatically as previously described, Wills-Karp, M. et al. Science 282, 2258, 1998) with 3×10⁴ P. aeruginosa (mucoid strain M57-15) entrapped in 50-200 μM agarose beads. Beginning 1 day prior to such challenge, mice were treated with an LXA₄ analogue (15-epi-16-[p-fluorophenoxy LXA₄] methyl ester) by oral administration, through placing 10 μg/ml of the analogue in the drinking water (Gewirtz, A. T. et al. J Immunol 168, 5260, 2002), supplemented with a single daily intravenous dose of 1 μg by tail vein. Controls received trace volumes (identical to that administered to lipoxin analogue-treated mice) of ethanol in drinking water and intravenously. Mice were weighed daily. Five days after P. aeruginosa/agar bead challenge, mice were sacrificed, BAL was performed, and lungs were harvested for measurement of lung Pseudomonas burden and myeloperoxidase by standard techniques (van Heeckeren, A. M. et al Lab Anim 36, 291, 2002; Wills-Karp, M. et al. Science 282, 2258, 1998; Andrews, P. C. et al. Anal Biochem 127, 346, 1982). Animal care was provided in accordance with National Institutes of Health guidelines. This was done in order to model an additional immunological feature of individuals with cystic fibrosis and P. aeruginosa colonization/infection: potent adaptive immune responses, marked by anti-pseudomonal antibodies and T cells reactive with pseudomonal proteins.

C57/B16 mice were challenged intratracheally (non-traumatically as previously described [Science 282:2258, 1998; Nature Immunol 1:221,2000]) with 3×10⁴ P. aeruginosa (mucoid strain M57-15) entrapped in agar beads (70-200 uM). Beginning 1 day prior to such challenge, mice were treated with an LX analogue (15-epi-16-(p Fluorophenoxy LXA₄ methyl ester) by oral administration, through placing 10 ug/ml of the LX analogue in the drinking water (J Immunol 168:5260, 2002), supplemented with a single daily intravenous dose of 1 ug by tail vein. LX analogues are stored in 95% ethanol. Controls received a (trace) volume of 95% ethanol identical to that administered (by drinking water and intravenously) to LX analogue-treated mice. Mice were weighed daily. Five days after P. aeruginosa/agar bead challenge, mice were sacrificed, bronchoalveolar lavage was performed, and lungs were harvested for measurement of lung Pseudomonas burden and myeloperoxidase (as a measure of tissue neutrophil accumulation.

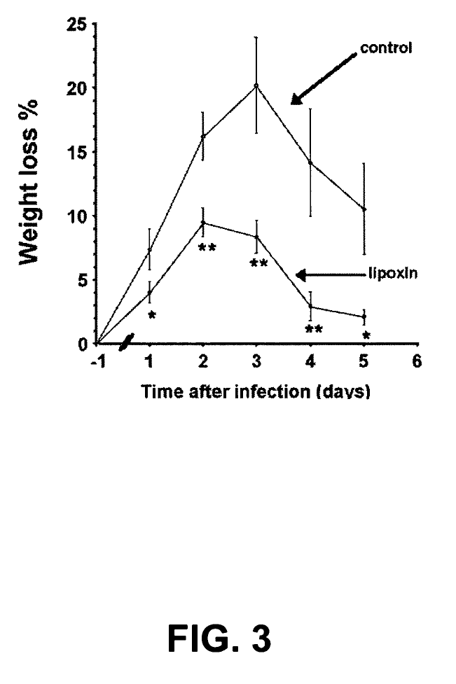

As shown in FIG. 3, mice treated with LX analogue had less weight loss, along with earlier and more complete recovery of lost weight, than did controls. FIG. 3 shows that LX analogue treatment ameliorates disease course in mice challenged with P. aeruginosa. C57BL/6 mice, challenged intratracheally with mucoid P. aeruginosa entrapped in agarose beads, were treated with LXA₄ analogue or vehicle control.

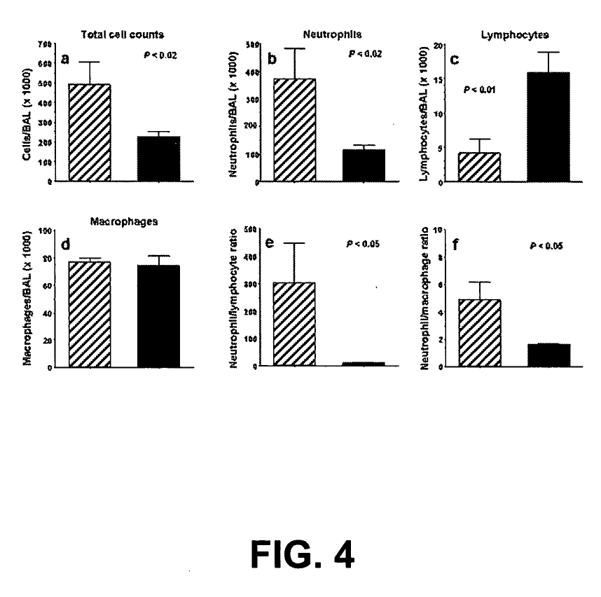

Analysis of cells present in bronchoalveolar lavage revealed (FIG. 4): (a) significant suppression of total cell counts; (b) significant inhibition of airway neutrophilia; (c) a significant increase in airway lymphocyte counts (despite the drop in the total number of cells); (d) an increase in the percentage of airway macrophages (obscured by the drop in the total number of cells); and (e) clear evidence of a shift from “acute” neutrophilic inflammation to “chronic” inflammation, marked by a significant decrease in neutrophil/lymphocyte and neutrophil/macrophage ratios in the airway. FIG. 4 shows that LX analogue treatment leads to suppression of neutrophilic inflammation and a shift to “chronic” inflammation in the airways in mice challenged with mucoid P. aeruginosa.

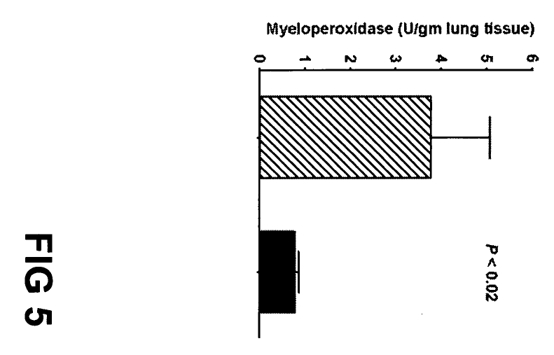

Analysis of whole lung myeloperoxidase levels was performed by standard techniques (Anal Biochem 127:346, 1982; J Immunol 161:474, 1998) in order to assess neutrophil accumulation in the lung parenchyma itself. As shown in FIG. 5, LX analogue therapy significantly reduced neutrophil accumulation in the lung parenchyma as well as the airway. FIG. 5 demonstrates that LX analogue treatment leads to suppression of neutrophil accumulation in the lung parenchyma after challenge with P. aeruginosa.

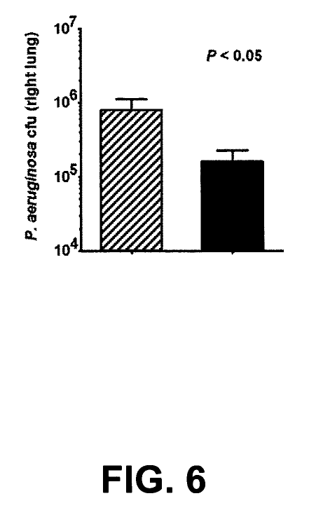

Analysis of whole lung P. aeruginosa burden by standard, quantitative microbiological techniques (FIG. 6) revealed that inhibition of the ineffective neutrophilic inflammatory response by lipoxin analogue led to a decrease in bacterial burden in the lung. FIG. 6 shows that LX analogue treatment leads to a decrease in lung bacterial burden after challenge with P. aeruginosa.

Since modifications will be apparent to those of skill in the art, it is intended that the subject matter claimed herein be limited only by the scope of the appended claims.

1. A method for the treatment of a subject with cystic fibrosis related disease comprising: administering to said subject a composition comprising lipoxin or lipoxin analogue in an amount sufficient to suppress or modulate airway neutrophilic inflammation.

2. A method for the treatment of a subject with cystic fibrosis related disease comprising: administering to said subject a composition containing lipoxin or lipoxin analogue in an amount sufficient to suppress or modulate airway neutrophilic inflammation.

3. A method for the treatment of a subject with cystic fibrosis related disease consisting of: administering to said subject a composition containing lipoxin or lipoxin analogue in an amount sufficient to suppress or modulate airway neutrophilic inflammation.

4. A method of claim 1 wherein said composition further comprises a pharmaceutically acceptable carrier.

5. A method of claim 1 wherein said composition contains a lipoxin molecule selected from a list consisting of: lipoxin A4 (LXA4), lipoxin B4 (LXB4), 15-epi-LXA4, 15-epi-LXB4, LXA5, 15-epi-LXA5, LXB5 and 15-epi-LXB5.

6. A method of claim 1 wherein said composition contains an ester, amide or carboxylic salt derivative of a lipoxin molecule selected from a list consisting of: lipoxin A4 (LXA4), lipoxin B4 (LXB4), 15-epi-LXA4, 15-epi-LXB4, LXA5, 15-epi-LXA5, LXB5 and 15-epi-LXB5.

7. A method of claim 1 wherein said composition contains a lipoxin analogue that has a structure that is similar to a natural lipoxin selected from a list consisting of: lipoxin A4 (LXA4), lipoxin B4 (LXB4), 15-epi-LXA4, 15-epi-LXB4, LXA5, 15-epi-LXA5, LXB5 and 15-epi-LXB5.

8. A method of claim 1 wherein said composition contains an ester, amide or carboxylic salt derivative of a lipoxin analogue that has a structure that is similar to a natural lipoxin selected from a list consisting of: lipoxin A4 (LXA4), lipoxin B4 (LXB4), 15-epi-LXA4, 15-epi-LXB4, LXA5, 15-epi-LXA5, LXB5 and 15-epi-LXB5.

9. A method of claim 1 wherein said composition contains a lipoxin analogue that has the following structure 1, 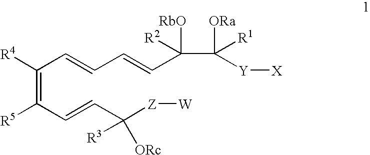
wherein:
X is selected from the group consisting of: —C(O)-A, —SO2-A, —PO(OR)-A, where A is hydroxy, alcoxy, aryloxy, amino, alkylamino, dialkylamino, or —OM, where M is a cation selected from the group consisting of ammonium, tetra-alkyl ammonium, Na, K, Mg, and Zn,
Y and Z are linkers independently selected from the group consisting of a chain of up to 20 atoms and a ring containing up to 20 atoms, provided that Y and Z can independently include one or more nitrogen, oxygen, sulfur or phosphorous atoms, and further provided that Y and Z can independently include one or more substituents selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that Y and Z can also contain one or more fused carbocyclic, heterocyclic, aryl or heteroaryl rings, and provided that linkers Y and Z are connected to the adjacent C(R)OR group via a carbon atom or a C-heteroatom bond where the heteroatom is oxygen, sulfur, phosphorous or nitrogen;
W is selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.
Ra, Rb and Rc, are independently selected from the group consisting of hydrogen, alkyl, aryl, heteroaryl, acyl, silyl, alcoxyacyl and aminoacyl;
R¹, R² and R³ are independently selected from the group consisting of hydrogen, alkyl, aryl and heteroaryl, provided that R¹, R² and R³ can independently be connected to linkers Y or Z;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond.

10. A method of claim 1 wherein said composition contains a lipoxin analogue that has the following structure 2 or 3, 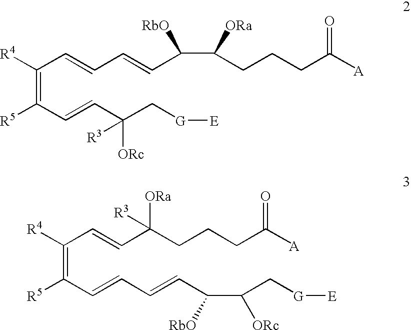
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
the stereochemistry at C-15 can be R or S.

11. A method of claim 1 wherein said composition contains a lipoxin analogue that has the following structure 4, 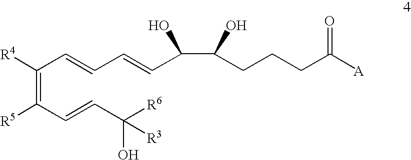
wherein:
A is selected from a group consisting of: hydroxy, methoxy, alcoxy, aryloxy, amino, alkylamino, dialkylamino, alkoxyamino, hydroxyamino,
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³and R⁶ may be connected together to form a carbocyclic, or heterocyclic ring of 3 to 8 atoms, including rings containing O, S and N atoms; and
the stereochemistry at C-15 can be R or S.

12. A method of claim 1 wherein said composition contains a lipoxin analogue that has the following structure 5, 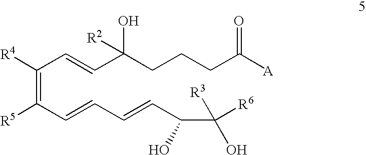
wherein:
A is selected from a group consisting of: hydroxy, methoxy, alcoxy, aryloxy, amino, alkylamino, dialkylamino, alkoxyamino, hydroxyamino,
R² and R³ are independently selected from the group consisting of hydrogen, alkyl, aryl and heteroaryl;
R⁴ and R⁵are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
R⁶ is selected from a group consisting of: methyl, alkyl, alkenyl, alkynyl, allyl, allenyl, alkoxyalkyl, aryloxyalkyl, heteroaryloxy, fluoroalkyl, cycloalkyl, aryl and heteroaryl. Groups R³ and R⁶ may be connected together to form a carbocyclic, or heterocyclic ring of 3 to 8 atoms, including rings containing O, S and N atoms; and
the stereochemistry at C-15 can be R or S.

13. A method of claim 1 wherein said composition contains a lipoxin analogue that has the following structure 6 or 7, 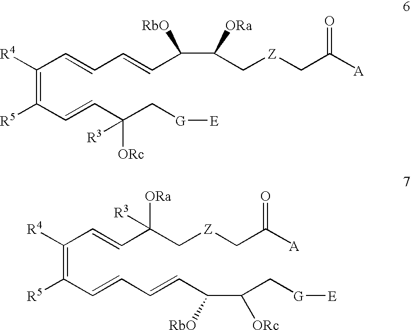
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Z is independently selected from the group consisting of: —O—, —S(O)—, —S(O)₂—, —C(═O)—, —N(Rd)-, —N(CORd)-, —N(SORd)-, —N(SO₂Rd)-, where Rd is alkyl, aryl, alcoxy, amido or —(CXY)—, where X and Y are hydrogen, halo, hydroxy, alcoxy, amino, amido, sulfonamido, alkyl, aryl, or acyl, and provided that X and Y can be joined together to form a carbocyclic or heterocyclic ring;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
the stereochemistry at C-15 can be R or S.

14. A method of claim 1 wherein said composition contains contains 15-epi-16-(p-fluorophenoxy) LXA4 methyl ester, of formula:

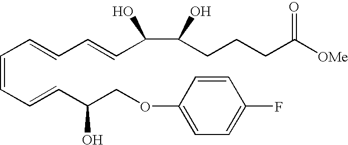

15. A method of claim 1 wherein said composition contains contains 15-11-dehydro-15-epi-16-(p-fluorophenoxy) LXA4 methyl ester, of formula:

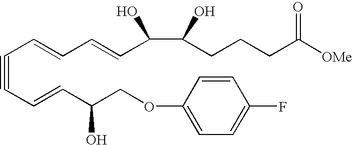

16. A composition containing a lipoxin analogue that has the following structure 1, 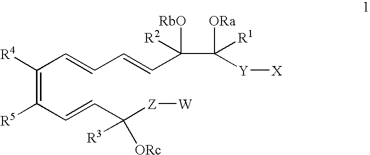
wherein:
X is selected from the group consisting of: —C(O)-A, —SO2-A, —PO(OR)-A, where A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Y and Z are linkers independently selected from the group consisting of a chain of up to 20 atoms and a ring containing up to 20 atoms, provided that Y and Z can each independently include one or more nitrogen, oxygen, sulfur or phosphorous atoms, and further provided that Y and Z can each independently include one or more substituents selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl; and further provided that Y and Z can also contain one or more fused carbocyclic, heterocyclic, aryl or heteroaryl rings, and provided that linkers Y and Z are connected to the adjacent C(R)OR group via a carbon atom or a C-heteroatom bond where the heteroatom is oxygen, sulfur, phosphorous or nitrogen;
W is selected from the group consisting of hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido;
Ra, Rb and Rc, are each independently selected from the group consisting of hydrogen, alkyl, aryl, heteroaryl, acyl, silyl, alcoxyacyl and aminoacyl;
R¹, R² and R³ are each independently selected from the group consisting of hydrogen, alkyl, aryl and heteroaryl, provided that R¹, R² and R³ can independently be connected to linkers Y or Z;
R⁴ and R⁵ are each independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond.

17. A composition containing a lipoxin analogue that has the following structure 2 or 3, 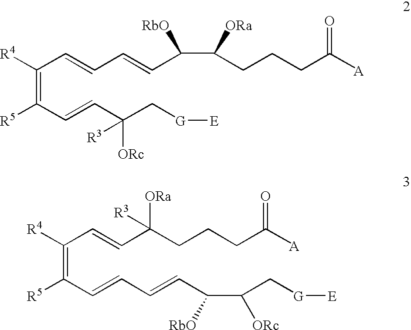
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Ra, Rb and Rc, are each independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl and heteroaryl;
E is selected from a group consisting of hydrogen, alkyl, alkenyl, alkynyl, aryl and heteroaryl;
the stereochemistry at C-15 can be R or S.

18. A composition containing a lipoxin analogue that has the following structure 6 or 7, 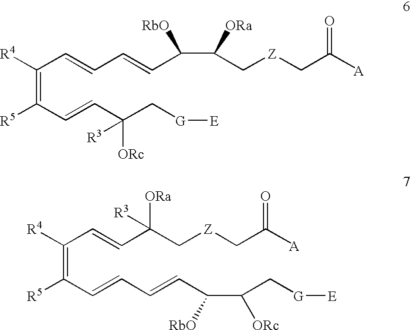
wherein:
A is hydroxy, alcoxy, aryloxy, amino, alkylamino, dialkylamino, or —OM, where M is a cation selected from the group consisting of ammonium, tetra-alkyl ammonium, Na, K, Mg, and Zn;
Z is independently selected from the group consisting of: —O—, —S(O)—, —S(O)₂—,
—C(═O)—, —N(Rd)-, —N(CORd)-, —N(SORd)-, —N(SO₂Rd)-, where Rd is alkyl, aryl, alcoxy, amido or —(CXY)—, where X and Y are hydrogen, halo, hydroxy, alcoxy, amino, amido, sulfonamido, alkyl, aryl, or acyl, and provided that X and Y can be joined together to form a carbocyclic or heterocyclic ring;
Ra, Rb and Rc, are independently selected from the group consisting of hydrogen, alkyl, aryl, heteroaryl, acyl, silyl, alcoxyacyl and aminoacyl;
R³ is selected from the group consisting of hydrogen, alkyl, aryl and heteroaryl;
R⁴ and R⁵ are independently selected from the group consisting of hydrogen, alkyl, alkenyl, alkynyl, aryl, heteroaryl, fluoro, and provided that R⁴ and R⁵ can be joined together to form a carbocyclic, heterocyclic or aromatic ring, and further provided that R⁴ and R⁵ can be replaced by a bond to form a triple bond;
G is selected from a group consisting of oxygen, alkyl, alkenyl, alkynyl, aryl, heteroaryl
E is selected from a group consisting of hydrogen, alkyl, alkenyl, alkynyl, aryl or heteroaryl;
the stereochemistry at C-15 can be R or S.

19. A method of claim 1, wherein a lipoxin or lipoxin analogue is used in combination with a therapeutic agent in an amount sufficient to treat prevent, or modulate neutrophilic airway inflammatory responses in subjects with cystic fibrosis related disease.
